(19) United States
(12) Patent Application Publication
(10) Pub. No.: US 2012/0214722 A1
(43) Pub. Date: Aug. 23, 2012

(54) TREATMENT SOLUTION FOR PREVENTING PATTERN COLLAPSE IN METAL FINE STRUCTURE BODY, AND PROCESS FOR PRODUCTION OF METAL FINE STRUCTURE BODY USING SAME

(75) Inventors: Masaru Ohto, Chiba (JP); Hiroshi Matsunaga, Tokyo (JP); Kenji Yamada, Tokyo (JP)

(73) Assignee: MITSUBISHI GAS CHEMICAL COMPANY INC., Tokyo (JP)

(21) Appl. No.: 13/502,867

(22) PCT Filed: Oct. 19, 2010
(86) PCT No.: PCT/JP2010/068396

§ 371 Date: Apr. 19, 2012

(30) Foreign Application Priority Data
Oct. 22, 2009 (JP) 2009-243488
Mar. 18, 2010 (JP) 2010-062936

(51) Int. Cl. C11D 7/60; C07C 229/20; C07C 291/04; C07C 211/63

(52) U.S. Cl. 510/175; 564/291; 562/574; 564/297

(57) ABSTRACT

There are provided a processing liquid for suppressing pattern collapse of a fine metal structure, containing at least one member selected from the group consisting of an ammonium halide having a fluoroalkyl group, a betaine compound having a fluoroalkyl group, and an amine oxide compound having a fluoroalkyl group, and a method for producing a fine metal structure using the same.

### TECHNICAL FIELD

The present invention relates to a processing liquid for suppressing pattern collapse of a fine metal structure, and a method for producing a fine metal structure using the same.

### BACKGROUND ART

The photolithography technique has been employed as a formation and processing method of a device having a fine structure used in a wide range of fields of art including a semiconductor device, a circuit board and the like. In these fields of art, reduction of size, increase of integration degree and increase of speed of a semiconductor device considerably proceed associated with the highly sophisticated demands on capabilities, which bring about continuous miniaturization and increase of aspect ratio of the resist pattern used for photolithography. However, the progress of miniaturization of the resist pattern causes pattern collapse as a major problem.

It has been known that upon drying a resist pattern from a processing liquid used in wet processing (which is mainly a rinsing treatment for washing away the developer solution) after developing the resist pattern, the collapse of the resist pattern is caused by the stress derived by the surface tension of the processing liquid. For preventing the collapse of the resist pattern, such methods have been proposed as a method of replacing the rinsing liquid by a liquid having a low surface tension using a nonionic surfactant, a compound soluble in an alcohol solvent, or the like and drying (see, for example, Patent Documents 1 and 2), and a method of hydrophobizing the surface of the resist pattern (see, for example, Patent Document 3).

In a fine structure formed of a metal, a metal nitride, a metal oxide or the like (which may be hereinafter referred to as a fine metal structure, and a metal, a metal nitride, a metal oxide or the like may be hereinafter referred totally as a metal) by the photolithography technique, the strength of the metal itself constituting the structure is larger than the strength of the resist pattern itself or the bonding strength between the resist pattern and the substrate, and therefore, the collapse of the structure pattern is hard to occur as compared to the resist pattern. However, associated with the progress of reduction of size, increase of integration degree and increase of speed of a semiconductor device and a micromachine, the pattern collapse of the structure is becoming a major problem due to miniaturization and increase of aspect ratio of the resist pattern. The fine metal structure has a surface state that is totally different from that of the resist pattern, which is an organic material, and therefore, there is no effective measure for preventing the pattern collapse of the structure. Accordingly, the current situation is that the degree of freedom on designing the pattern for producing a semiconductor device or a micromachine with reduced size, increased integration degree and increased speed is considerably impaired since the pattern is necessarily designed for preventing the pattern collapse.

### PRIOR ART DOCUMENTS

### Patent Documents

Patent Document 1: JP-A-2004-184648

Patent Document 2: JP-A-2005-309260

Patent Document 3: JP-A-2006-163314

### SUMMARY OF THE INVENTION

### Problems to be Solved by the Invention

As described above, the current situation is that no effective technique for suppressing pattern collapse has been known in the field of a fine metal structure, such as a semiconductor device and a micromachine.

The present invention has been developed under the circumstances, and an object thereof is to provide a processing liquid that is capable of suppressing pattern collapse of a fine metal structure, such as a semiconductor device and a micromachine, and a method for producing a fine metal structure using the same.

### Means for Solving the Problems

As a result of earnest investigations made by the inventors for achieving the object, it has been found that the object can be achieved with a processing liquid containing at least one member selected from an ammonium halide having a fluoroalkyl group, a betaine compound having a fluoroalkyl group, and an amine oxide compound having a fluoroalkyl group.

The present invention has been completed based on the finding. Accordingly, the gist of the present invention is as follows.

(1) A processing liquid for suppressing pattern collapse of a fine metal structure, containing at least one member selected from the group consisting of an ammonium halide having a fluoroalkyl group, a betaine compound having a fluoroalkyl group, and an amine oxide compound having a fluoroalkyl group.

(2) The processing liquid according to the item (1), wherein a content of the ammonium halide having a fluoroalkyl group, the betaine compound having a fluoroalkyl group, and the amine oxide compound having a fluoroalkyl group is from 10 ppm to 50%.

(3) The processing liquid according to the item (1) or (2), which further contains water.

(4) The processing liquid according to any one of the items (1) to (3), wherein the pattern of the fine metal structure contains at least one material selected from the group consisting of titanium nitride, tungsten, hafnium oxide, tantalum and titanium.

(5) A method for producing a fine metal structure, containing after wet etching or dry etching, a rinsing step using the processing liquid according to any one of the items (1) to (4).

(6) The method for producing a fine metal structure according to the item (5), wherein the fine metal structure contains at least one material selected from the group consisting of titanium nitride, tungsten, hafnium oxide, tantalum and titanium.

(7) The method for producing a fine metal structure according to the item (5) or (6), wherein the fine metal structure is a semiconductor device or a micromachine.

### Advantages of the Invention

According to the present invention, there are provided a processing liquid that is capable of suppressing pattern collapse of a fine metal structure, such as a semiconductor device and a micromachine, and a method for producing a fine metal structure using the same.

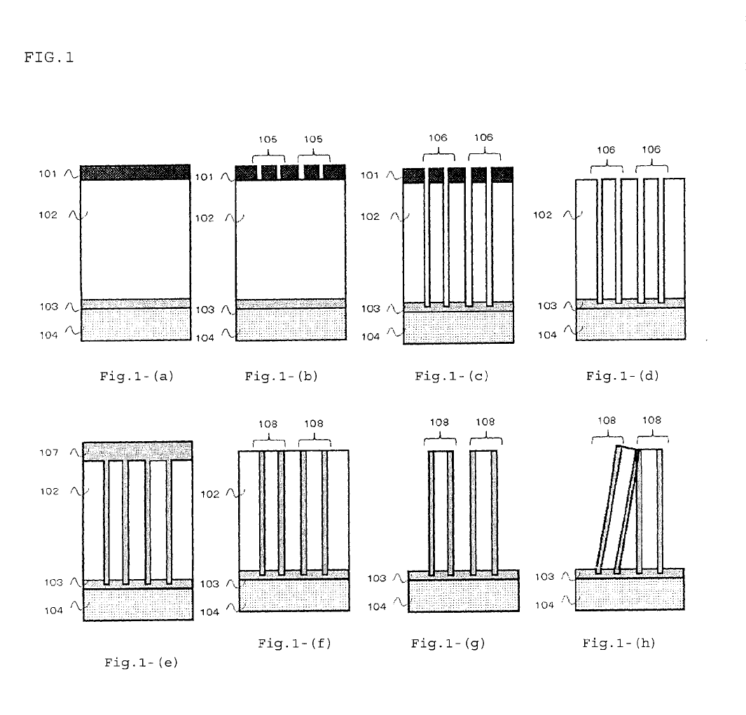

### BRIEF DESCRIPTION OF THE DRAWINGS

[FIG. 1] The figure includes schematic cross sectional views of each production steps of fine metal structures produced in Examples 1 to 45 and Comparative Examples 1 to 65.

### MODE FOR CARRYING OUT THE INVENTION

The processing liquid of the present invention is used for suppressing pattern collapse of a fine metal structure, and contains at least one member selected from an ammonium halide having a fluoroalkyl group, a betaine compound having a fluoroalkyl group, and an amine oxide compound having a fluoroalkyl group.

It is considered that the ammonium halide having a fluoroalkyl group, the betaine compound having a fluoroalkyl group, and the amine oxide compound having a fluoroalkyl group used in the processing liquid of the present invention are adsorbed on the metal material used in the pattern of the fine metal structure, thereby hydrophobizing the surface of the pattern. The hydrophobization in this case means that the contact angle of the metal surface having been processed with the processing liquid of the present invention with respect to water is 70° or more.

The fluoroalkyl group referred in the present invention is a perfluoroalkyl group, and the perfluoroalkyl group means such a group that all hydrogen atoms of an alkyl group are replaced by fluorine atoms. The fluoroalkyl group preferably has from 1 to 6 carbon atoms.

Examples of the ammonium halide having a fluoroalkyl group include Fluorad FC-135, a product name (produced by Sumitomo 3M, Ltd.), Ftergent 300, a product name (produced by Neos Co., Ltd.), Ftergent 310, a product name (produced by Neos Co., Ltd.) Surfron S-121, a product name (produced by AGC Seimi Chemical Co., Ltd.) and Surfron S-221, a product name (produced by AGC Seimi Chemical Co., Ltd.), and in particular, Surfron S-221, a product name (produced by AGC Seimi Chemical Co., Ltd.) is preferred.

Examples of the betaine compound having a fluoroalkyl group include Ftergent 400S, a product name (produced by Neos Co., Ltd.), Surfron S-131, a product name (produced by AGC Seimi Chemical Co., Ltd.), Surfron S-132, a product name (produced by AGC Seimi Chemical Co., Ltd.) and Surfron S-231, a product name (produced by AGC Seimi Chemical Co., Ltd.), and in particular, Surfron S-231, a product name (produced by AGC Seimi Chemical Co., Ltd.) is preferred.

Examples of the amine oxide compound having a fluoroalkyl group include Surfron S-141, a product name (produced by AGC Seimi Chemical Co., Ltd.) and Surfron S-241, a product name (produced by AGC Seimi Chemical Co., Ltd.), and in particular, Surfron S-241, a product name (produced by AGC Seimi Chemical Co., Ltd.) is preferred.

The processing liquid of the present invention preferably further contains water and is preferably an aqueous solution. Preferred examples of the water include water, from which metallic ions, organic impurities, particles and the like are removed by distillation, ion exchange, filtering, adsorption treatment or the like, and particularly preferred examples thereof include pure water and ultrapure water.

The processing liquid of the present invention contains at least one member selected from the ammonium halide having a fluoroalkyl group, the betaine compound having a fluoroalkyl group, and the amine oxide compound having a fluoroalkyl group, preferably contains water, and may contain various kinds of additives that are ordinarily used in processing liquids in such a range that does not impair the advantages of the processing liquid.

The content of the ammonium halide having a fluoroalkyl group, the betaine compound having a fluoroalkyl group, and the amine oxide compound having a fluoroalkyl group in the processing liquid of the present invention (which is the total content in the case where plural compounds are contained) is preferably from 10 ppm to 50%, more preferably 30% or less, and further preferably 10% or less, and in consideration of handleability, economy and foaming, the content is still further preferably 5% or less, furthermore from 10 to 2,000 ppm, and particularly preferably from 10 to 1,000 ppm. In the case where the compounds do not have sufficient solubility in water to cause phase separation, an organic solvent, such as an alcohol, may be added, and an acid or an alkali may be added to enhance the solubility. Even in the case where the processing liquid is simply turbid white without phase separation, the processing liquid may be used in such a range that does not impair the advantages of the processing liquid, and may be used while stirring to make the processing liquid homogeneous. Furthermore, for avoiding the white turbidity of the processing liquid, the processing liquid may be used after adding an organic solvent, such as an alcohol, an acid or an alkali thereto as similar to the above case.

The processing liquid of the present invention may be used favorably for suppressing pattern collapse of a fine metal structure, such as a semiconductor device and a micromachine. Preferred examples of the pattern of the fine metal structure include ones containing at least one member selected from TiN (titanium nitride), W (tungsten), HfO₂ (hafnium oxide), Ta (tantalum) and Ti (titanium).

The fine metal structure may be patterned on an insulating film species, such as SiO₂ (a silicon oxide film) and TEOS (a tetraethoxy ortho silane), in some cases, or the insulating film species is contained as apart of the fine metal structure in some cases.

The processing liquid of the present invention can exhibit excellent pattern collapse suppressing effect to not only an ordinary fine metal structure, but also a fine metal structure with further miniaturization and higher aspect ratio. The aspect ratio referred herein is a value calculated from (height of pattern/width of pattern), and the processing liquid of the present invention may exhibit excellent pattern collapse suppressing effect to a pattern that has a high aspect ratio of 3 or more, and further 7 or more. The processing liquid of the present invention has excellent pattern collapse suppressing effect to a finer pattern with a pattern size (pattern width) of 300 nm or less, further 150 nm or less, and still further 100 nm or less, and with a pattern size of 50 nm or less and a line/space ratio of 1/1, and similarly to a finer pattern with a pattern distance of 300 nm or less, further 150 nm or less, still further 100 nm or less, and still further 50 nm or less and a cylindrical hollow or cylindrical solid structure.

### Method for Producing Fine Metal Structure

The method for producing a fine metal structure of the present invention contains, after wet etching or dry etching, a rinsing step using the processing liquid of the present invention. More specifically, in the rinsing step, it is preferred that the pattern of the fine metal structure is made in contact with the processing liquid of the present invention by dipping, spray ejecting, spraying or the like, then the processing liquid is replaced by water, and the fine metal structure is dried. In the case where the pattern of the fine metal structure and the processing liquid of the present invention are in contact with each other by dipping, the dipping time is preferably from 10 seconds to 30 minutes, more preferably from 15 seconds to 20 minutes, further preferably from 20 seconds to 15 minutes, and particularly preferably from 30 seconds to 10 minutes, and the temperature condition is preferably from 10 to 60° C., more preferably from 15 to 50° C., further preferably from 20 to 40° C., and particularly preferably from 25 to 40° C. The pattern of the fine metal structure may be rinsed with water before making in contact with the processing liquid of the present invention. The contact between the pattern of the fine metal structure and the processing liquid of the present invention enables suppression of collapse of the pattern, in which a pattern is in contact with the adjacent pattern, through hydrophobization of the surface of the pattern.

The processing liquid of the present invention may be applied widely to a production process of a fine metal structure irrespective of the kind of the fine metal structure, as far as the production process has a step of wet etching or dry etching, then a step of wet processing (such as etching, cleaning or rinsing for washing the cleaning liquid), and then a drying step. For example, the processing liquid of the present invention may be favorably used after the etching step in the production process of a semiconductor device or a micromachine, for example, (i) after wet etching of an insulating film around an electroconductive film in the production of a DRAM type semiconductor device (see, for example, JP-A-2000-196038 and JP-A-2004-288710), (ii) after a rinsing step for removing contamination formed after dry etching or wet etching upon processing a gate electrode in the production of a semiconductor device having a transistor with a fin in the form of strips (see, for example, JP-A-2007-335892), and (iii) after a rinsing step for removing contamination formed after etching for forming a cavity by removing sacrifice layer formed of an insulating film through a through hole in an electroconductive film upon forming a cavity of a micromachine (electrodynamic micromachine) (see, for example, JP-A-2009-122031).

### EXAMPLE

The present invention will be described in more detail with reference to examples and comparative examples below, but the present invention is not limited to the examples.

### Preparation of Processing Liquid

Processing liquids for suppressing pattern collapse of a fine metal structure were prepared according the formulation compositions (% by mass) shown in Table 1. The balance is water.

TABLE 1

| | Kind | Content | |
|---|---|---|---|
| Processing liquid 1 | Surfron S-221 *¹ | 50% | |
| Processing liquid 2 | Surfron S-221 *¹ | 2% | |
| Processing liquid 3 | Surfron S-221 *¹ | 1,000 | ppm |
| Processing liquid 4 | Surfron S-231 *² | 20% | |
| Processing liquid 5 | Surfron S-231 *² | 1 | ppm |
| Processing liquid 6 | Surfron S-231 *² | 10 | ppm |
| Processing liquid 7 | Surfron S-241 *³ | 10% | |
| Processing liquid 8 | Surfron S-241 *³ | 1% | |
| Processing liquid 9 | Surfron S-241 *³ | 50 | ppm |

*¹ “Surfron S-221 (trade name), produced by AGC Seimi Chemical Co., Ltd., perfluoroalkyltrialkylammonium halide
*² “Surfron S-231 (trade name), produced by AGC Seimi Chemical Co., Ltd., perfluoroalkyl betaine
*³ “Surfron S-241 (trade name), produced by AGC Seimi Chemical Co., Ltd., perfluoroalkylamine oxide

### Examples 1 to 9

As shown in FIG. 1(a), silicon nitride 103 (thickness: 100 nm) and silicon oxide 102 (thickness: 1,200 nm) were formed as films on a silicon substrate 104, then a photoresist 101 was formed, and the photoresist 101 was exposed and developed, thereby forming a circular and ring-shaped opening 105 (diameter: 125 nm, distance between circles: 50 nm), as shown in FIG. 1(b). The silicon oxide 102 was etched by dry etching with the photoresist 101 as a mask, thereby forming a cylindrical hole 106 reaching the layer of silicon nitride 103, as shown in FIG. 1(c). The photoresist 101 was then removed by ashing, thereby providing a structure having the silicon oxide 102 with the cylindrical hole 106 reaching the layer of silicon nitride 103, as shown in FIG. 1(d). The cylindrical hole 106 of the resulting structure was filled with tungsten as a metal 107 (FIG. 1(e)), and an excessive portion of the metal (tungsten) 107 was removed by chemical mechanical polishing (CMP), thereby providing a structure having the silicon oxide 102 with a cylindrical hollow of the metal (tungsten) 108 embedded therein, as shown in FIG. 1(f). The silicon oxide 102 of the resulting structure was removed by dissolving with a 0.5% hydrofluoric acid aqueous solution (by dipping at 25° C. for 1 minute), and then the structure was processed by making into contact with pure water, the processing liquids 1 to 18 (by dipping at 30° C. for 10 minutes), and pure water in this order, followed by drying, thereby providing a structure shown in FIG. 1(g).

The resulting structure had a fine structure with a chimney pattern containing cylindrical hollows of the metal (tungsten) (diameter: 125 nm, height: 1,200 nm (aspect ratio: 9.6), distance between the cylindrical hollows: 50 nm), and 70% or more of the pattern was not collapsed.

The pattern collapse was observed with “FE-SEM S-5500 (model number)”, produced by Hitachi High-Technologies Corporation, and the collapse suppression ratio was a value obtained by calculating the ratio of pattern not collapsed in the total pattern. Cases where the collapse suppression ratio was 50% or more were determined as “passed”. The processing liquids, the processing methods and the results of collapse suppression ratios in the examples are shown in Table 3.

### Comparative Example 1

A structure shown in FIG. 1(g) was obtained in the same manner as in Example 1 except that after removing the silicon oxide 102 of the structure shown in FIG. 1(f) by dissolving with hydrofluoric acid, the structure was processed only with pure water. 50% or more of the pattern of the resulting structure was collapsed as shown in FIG. 1(h) (which indicated a collapse suppression ratio of less than 50%). The processing liquid, the processing method and the result of collapse suppression ratio in Comparative Example 1 are shown in Table 3.

### Comparative Examples 2 to 14

Structures shown in FIG. 1(g) of Comparative Examples 2 to 14 were obtained in the same manner as in Example 1 except that after removing the silicon oxide 102 of the structure shown in FIG. 1(f) by dissolving with hydrofluoric acid and then processed with pure water, the structures were processed with the comparative liquids 1 to 13 shown in Table 2 instead of the processing liquid 1. 50% or more of the pattern of the resulting structures was collapsed as shown in FIG. 1(h). The comparative liquids, the processing methods and the results of collapse suppression ratios in the comparative examples are shown in Table 3.

TABLE 2

| | Name of substance |
|---|---|
| Comparative liquid 1 | isopropyl alcohol |
| Comparative liquid 2 | diethylene glycol monomethyl ether |
| Comparative liquid 3 | dimethylacetamide |
| Comparative liquid 4 | ammonium halide perfluoroalkylsulfonate *¹ |
| Comparative liquid 5 | perfluoroalkylcarbonate salt *² |
| Comparative liquid 6 | ethylene oxide adduct of 2,4,7,9-tetramethyl-5- |
| | decine-4,7-diol *³ |
| Comparative liquid 7 | 2,4,7,9-tetramethyl-5-decine-4,7-diol *⁴ |
| Comparative liquid 8 | dodecyltrimethylammonium chloride (number of |
| | carbon atoms of alkyl group: 12) *⁵ |
| Comparative liquid 9 | polyoxyethylene polyoxypropylene block |
| | polymer *⁶ |
| Comparative liquid 10 | 1-decyl-3-methylimidazolium chloride (number of |
| | carbon atoms of alkyl group: 10) |
| Comparative liquid 11 | 1-dodecylpyridinium chloride (number of carbon |
| | atoms of alkyl group: 12) |
| Comparative liquid 12 | 1-decyl-3-methylimidazolium chloride (number of |
| | carbon atoms of alkyl group: 10) |
| Comparative liquid 13 | dimethyldodecylamine oxide (number of carbon |
| | atoms of alkyl group: 12) |

*¹ “Fluorad FC-93”, a trade name, produced by 3M Corporation, 0.01% aqueous solution
*² “Surfron S-111”, a trade name, produced by AGC Seimi Chemical Co., Ltd., 0.01% aqueous solution
*³ “Surfynol 420”, a trade name, produced by Nisshin Chemical Industry Co., Ltd., 0.01% aqueous solution
*⁴ “Surfynol 104”, a trade name, produced by Nisshin Chemical Industry Co., Ltd., 0.01% aqueous solution
*⁵ “Catiogen TML”, a trade name produced by Dai-ichi Kogyo Seiyaku Co., Ltd., 0.01% aqueous solution
*⁶ “Epan 420”, a trade name produced by Dai-ichi Kogyo Seiyaku Co., Ltd., 0.01% aqueous solution

TABLE 3

| | Processing method | Collapse suppression ratio *¹ | Pass or fail |
|---|---|---|---|
| Example 1 | pure water → processing liquid 1 → pure water → drying | 80% or more | pass |
| Example 2 | pure water → processing liquid 2 → pure water → drying | 80% or more | pass |
| Example 3 | pure water → processing liquid 3 → pure water → drying | 70% or more | pass |
| Example 4 | pure water → processing liquid 4 → pure water → drying | 80% or more | pass |
| Example 5 | pure water → processing liquid 5 → pure water → drying | 80% or more | pass |
| Example 6 | pure water → processing liquid 6 → pure water → drying | 70% or more | pass |
| Example 7 | pure water → processing liquid 7 → pure water → drying | 90% or more | pass |
| Example 8 | pure water → processing liquid 8 → pure water → drying | 90% or more | pass |
| Example 9 | pure water → processing liquid 9 → pure water → drying | 80% or more | pass |
| Comparative Example 1 | pure water → drying | less than 50% | fail |
| Comparative Example 2 | pure water → comparative liquid 1 → pure water → drying | less than 50% | fail |
| Comparative Example 3 | pure water → comparative liquid 2 → pure water → drying | less than 50% | fail |
| Comparative Example 4 | pure water → comparative liquid 3 → pure water → drying | less than 50% | fail |
| Comparative Example 5 | pure water → comparative liquid 4 → pure water → drying | less than 50% | fail |
| Comparative Example 6 | pure water → comparative liquid 5 → pure water → drying | less than 50% | fail |
| Comparative Example 7 | pure water → comparative liquid 6 → pure water → drying | less than 50% | fail |
| Comparative Example 8 | pure water → comparative liquid 7 → pure water → drying | less than 50% | fail |
| Comparative Example 9 | pure water → comparative liquid 8 → pure water → drying | less than 50% | fail |
| Comparative Example 10 | pure water → comparative liquid 9 → pure water → drying | less than 50% | fail |
| Comparative Example 11 | pure water → comparative liquid 10 → pure water → drying | less than 50% | fail |
| Comparative Example 12 | pure water → comparative liquid 11 → pure water → drying | less than 50% | fail |
| Comparative Example 13 | pure water → comparative liquid 12 → pure water → drying | less than 50% | fail |
| Comparative Example 14 | pure water → comparative liquid 13 → pure water → drying | less than 50% | fail |

*¹ collapse suppression ratio = ((number of cylindrical hollows not collapsed)/(total number of cylindrical hollows)) × 100 (%)

### Examples 10 to 18

Structures shown in FIG. 1(g) were obtained in the same manner as in Examples 1 to 9 except that titanium nitride was used as the metal 107 instead of tungsten. The resulting structures had a fine structure with a pattern containing cylindrical hollows 108 of the metal (titanium nitride) (diameter: 125 nm, height: 1,200 nm (aspect ratio: 9.6), distance between the cylindrical hollows: 50 nm), and 70% or more of the pattern was not collapsed. The processing liquids, the processing methods and the results of collapse suppression ratios in the examples are shown in Table 4.

### Comparative Examples 15 to 27

Structures shown in FIG. 1(g) of Comparative Examples 15 to 27 were obtained in the same manner as in Comparative Examples 1 to 14 except that titanium nitride was used as the metal 107 instead of tungsten. 50% or more of the pattern of the resulting structures was collapsed as shown in FIG. 1(h). The processing liquids, the processing methods and the results of collapse suppression ratios in the examples are shown in Table 4.

TABLE 4

| | Processing method | Collapse suppression ratio *¹ | Pass or fail |
|---|---|---|---|
| Example 10 | pure water → processing liquid 1 → pure water → drying | 70% or more | pass |
| Example 11 | pure water → processing liquid 2 → pure water → drying | 70% or more | pass |
| Example 12 | pure water → processing liquid 3 → pure water → drying | 70% or more | pass |
| Example 13 | pure water → processing liquid 4 → pure water → drying | 80% or more | pass |
| Example 14 | pure water → processing liquid 5 → pure water → drying | 80% or more | pass |
| Example 15 | pure water → processing liquid 6 → pure water → drying | 70% or more | pass |
| Example 16 | pure water → processing liquid 7 → pure water → drying | 90% or more | pass |
| Example 17 | pure water → processing liquid 8 → pure water → drying | 90% or more | pass |
| Example 18 | pure water → processing liquid 9 → pure water → drying | 80% or more | pass |
| Comparative Example 15 | pure water → drying | less than 50% | fail |
| Comparative Example 16 | pure water → comparative liquid 1 → pure water → drying | less than 50% | fail |
| Comparative Example 17 | pure water → comparative liquid 2 → pure water → drying | less than 50% | fail |
| Comparative Example 18 | pure water → comparative liquid 3 → pure water → drying | less than 50% | fail |
| Comparative Example 19 | pure water → comparative liquid 4 → pure water → drying | less than 50% | fail |
| Comparative Example 20 | pure water → comparative liquid 5 → pure water → drying | less than 50% | fail |
| Comparative Example 21 | pure water → comparative liquid 6 → pure water → drying | less than 50% | fail |
| Comparative Example 22 | pure water → comparative liquid 7 → pure water → drying | less than 50% | fail |
| Comparative Example 23 | pure water → comparative liquid 8 → pure water → drying | less than 50% | fail |
| Comparative Example 24 | pure water → comparative liquid 9 → pure water → drying | less than 50% | fail |
| Comparative Example 25 | pure water → comparative liquid 10 → pure water → drying | less than 50% | fail |
| Comparative Example 26 | pure water → comparative liquid 11 → pure water → drying | less than 50% | fail |
| Comparative Example 27 | pure water → comparative liquid 12 → pure water → drying | less than 50% | fail |

*¹ collapse suppression ratio = ((number of cylindrical hollows not collapsed)/(total number of cylindrical hollows)) × 100 (%)

### Examples 19 to 27

Structures shown in FIG. 1(g) were obtained in the same manner as in Examples 1 to 9 except that hafnium oxide was used as the metal 107 instead of tungsten. The resulting structures had a fine structure with a pattern containing cylindrical hollows 108 of the metal (hafnium oxide) (diameter: 125 nm, height: 1,200 nm (aspect ratio: 9.6), distance between the cylindrical hollows: 50 nm), and 70% or more of the pattern was not collapsed. The processing liquids, the processing methods and the results of collapse suppression ratios in the examples are shown in Table 5.

### Comparative Examples 28 to 40

Structures shown in FIG. 1(g) of Comparative Examples 28 to 40 were obtained in the same manner as in Comparative Examples 1 to 14 except that hafnium oxide was used as the metal 107 instead of tungsten. 50% or more of the pattern of the resulting structures was collapsed as shown in FIG. 1(h). The processing liquids, the processing methods and the results of collapse suppression ratios in the examples are shown in Table 5.

TABLE 5

| | Processing method | Collapse suppression ratio *¹ | Pass or fail |
|---|---|---|---|
| Example 19 | pure water → processing liquid 1 → pure water → drying | 80% or more | pass |
| Example 20 | pure water → processing liquid 2 → pure water → drying | 80% or more | pass |
| Example 21 | pure water → processing liquid 3 → pure water → drying | 70% or more | pass |
| Example 22 | pure water → processing liquid 4 → pure water → drying | 80% or more | pass |
| Example 23 | pure water → processing liquid 5 → pure water → drying | 80% or more | pass |
| Example 24 | pure water → processing liquid 6 → pure water → drying | 70% or more | pass |
| Example 25 | pure water → processing liquid 7 → pure water → drying | 80% or more | pass |
| Example 26 | pure water → processing liquid 8 → pure water → drying | 80% or more | pass |
| Example 27 | pure water → processing liquid 9 → pure water → drying | 70% or more | pass |
| Comparative Example 28 | pure water → drying | less than 50% | fail |
| Comparative Example 29 | pure water → comparative liquid 1 → pure water → drying | less than 50% | fail |
| Comparative Example 30 | pure water → comparative liquid 2 → pure water → drying | less than 50% | fail |
| Comparative Example 31 | pure water → comparative liquid 3 → pure water → drying | less than 50% | fail |
| Comparative Example 32 | pure water → comparative liquid 4 → pure water → drying | less than 50% | fail |
| Comparative Example 33 | pure water → comparative liquid 5 → pure water → drying | less than 50% | fail |
| Comparative Example 34 | pure water → comparative liquid 6 → pure water → drying | less than 50% | fail |
| Comparative Example 35 | pure water → comparative liquid 7 → pure water → drying | less than 50% | fail |
| Comparative Example 36 | pure water → comparative liquid 8 → pure water → drying | less than 50% | fail |
| Comparative Example 37 | pure water → comparative liquid 9 → pure water → drying | less than 50% | fail |
| Comparative Example 38 | pure water → comparative liquid 10 → pure water → drying | less than 50% | fail |
| Comparative Example 39 | pure water → comparative liquid 11 → pure water → drying | less than 50% | fail |
| Comparative Example 40 | pure water → comparative liquid 12 → pure water → drying | less than 50% | fail |

*¹ collapse suppression ratio = ((number of cylindrical hollows not collapsed)/(total number of cylindrical hollows)) × 100 (%)

### Examples 28 to 36

Structures shown in FIG. 1(g) were obtained in the same manner as in Examples 1 to 9 except that tantalum was used as the metal 107 instead of tungsten. The resulting structures had a fine structure with a pattern containing cylindrical hollows 108 of the metal (tantalum) (diameter: 125 nm, height: 1,200 nm (aspect ratio: 9.6), distance between the cylindrical hollows: 50 nm), and 70% or more of the pattern was not collapsed. The processing liquids, the processing methods and the results of collapse suppression ratios in the examples are shown in Table 6.

### Comparative Examples 41 to 53

Structures shown in FIG. 1(g) of Comparative Examples 41 to 53 were obtained in the same manner as in Comparative Examples 1 to 14 except that tantalum was used as the metal 107 instead of tungsten. 50% or more of the pattern of the resulting structures was collapsed as shown in FIG. 1(h). The processing liquids, the processing methods and the results of collapse suppression ratios in the examples are shown in Table 6.

TABLE 6

| | Processing method | Collapse suppression ratio *¹ | Pass or fail |
|---|---|---|---|
| Example 28 | pure water → processing liquid 1 → pure water → drying | 80% or more | pass |
| Example 29 | pure water → processing liquid 2 → pure water → drying | 80% or more | pass |
| Example 30 | pure water → processing liquid 3 → pure water → drying | 70% or more | pass |
| Example 31 | pure water → processing liquid 4 → pure water → drying | 80% or more | pass |
| Example 32 | pure water → processing liquid 5 → pure water → drying | 80% or more | pass |
| Example 33 | pure water → processing liquid 6 → pure water → drying | 70% or more | pass |
| Example 34 | pure water → processing liquid 7 → pure water → drying | 80% or more | pass |
| Example 35 | pure water → processing liquid 8 → pure water → drying | 80% or more | pass |
| Example 36 | pure water → processing liquid 9 → pure water → drying | 70% or more | pass |
| Comparative Example 41 | pure water → drying | less than 50% | fail |
| Comparative Example 42 | pure water → comparative liquid 1 → pure water → drying | less than 50% | fail |
| Comparative Example 43 | pure water → comparative liquid 2 → pure water → drying | less than 50% | fail |
| Comparative Example 44 | pure water → comparative liquid 3 → pure water → drying | less than 50% | fail |
| Comparative Example 45 | pure water → comparative liquid 4 → pure water → drying | less than 50% | fail |
| Comparative Example 46 | pure water → comparative liquid 5 → pure water → drying | less than 50% | fail |
| Comparative Example 47 | pure water → comparative liquid 6 → pure water → drying | less than 50% | fail |
| Comparative Example 48 | pure water → comparative liquid 7 → pure water → drying | less than 50% | fail |
| Comparative Example 49 | pure water → comparative liquid 8 → pure water → drying | less than 50% | fail |
| Comparative Example 50 | pure water → comparative liquid 9 → pure water → drying | less than 50% | fail |
| Comparative Example 51 | pure water → comparative liquid 10 → pure water → drying | less than 50% | fail |
| Comparative Example 52 | pure water → comparative liquid 11 → pure water → drying | less than 50% | fail |
| Comparative Example 53 | pure water → comparative liquid 12 → pure water → drying | less than 50% | fail |

*¹ collapse suppression ratio = ((number of cylindrical hollows not collapsed)/(total number of cylindrical hollows)) × 100 (%)

### Examples 37 to 45

Structures shown in FIG. 1(g) were obtained in the same manner as in Examples 1 to 9 except that titanium was used as the metal 107 instead of tungsten. The resulting structures had a fine structure with a pattern containing cylindrical hollows 108 of the metal (titanium) (diameter: 125 nm, height: 1,200 nm (aspect ratio: 9.6), distance between the cylindrical hollows: 50 nm), and 70% or more of the pattern was not collapsed. The processing liquids, the processing methods and the results of collapse suppression ratios in the examples are shown in Table 7.

### Comparative Examples 53 to 65

Structures shown in FIG. 1(g) of Comparative Examples 53 to 65 were obtained in the same manner as in Comparative Examples 1 to 14 except that titanium was used as the metal 107 instead of tungsten. 50% or more of the pattern of the resulting structures was collapsed as shown in FIG. 1(h). The processing liquids, the processing methods and the results of collapse suppression ratios in the examples are shown in Table 7.

TABLE 7

| | Processing method | Collapse suppression ratio *¹ | Pass or fail |
|---|---|---|---|
| Example 37 | pure water → processing liquid 1 → pure water → drying | 70% or more | pass |
| Example 38 | pure water → processing liquid 2 → pure water → drying | 70% or more | pass |
| Example 39 | pure water → processing liquid 3 → pure water → drying | 70% or more | pass |
| Example 40 | pure water → processing liquid 4 → pure water → drying | 80% or more | pass |
| Example 41 | pure water → processing liquid 5 → pure water → drying | 80% or more | pass |
| Example 42 | pure water → processing liquid 6 → pure water → drying | 70% or more | pass |
| Example 43 | pure water → processing liquid 7 → pure water → drying | 80% or more | pass |
| Example 44 | pure water → processing liquid 8 → pure water → drying | 80% or more | pass |
| Example 45 | pure water → processing liquid 9 → pure water → drying | 70% or more | pass |
| Comparative Example 53 | pure water → drying | less than 50% | fail |
| Comparative Example 54 | pure water → comparative liquid 1 → pure water → drying | less than 50% | fail |
| Comparative Example 55 | pure water → comparative liquid 2 → pure water → drying | less than 50% | fail |
| Comparative Example 56 | pure water → comparative liquid 3 → pure water → drying | less than 50% | fail |
| Comparative Example 57 | pure water → comparative liquid 4 → pure water → drying | less than 50% | fail |
| Comparative Example 58 | pure water → comparative liquid 5 → pure water → drying | less than 50% | fail |
| Comparative Example 59 | pure water → comparative liquid 6 → pure water → drying | less than 50% | fail |
| Comparative Example 60 | pure water → comparative liquid 7 → pure water → drying | less than 50% | fail |
| Comparative Example 61 | pure water → comparative liquid 8 → pure water → drying | less than 50% | fail |
| Comparative Example 62 | pure water → comparative liquid 9 → pure water → drying | less than 50% | fail |
| Comparative Example 63 | pure water → comparative liquid 10 → pure water → drying | less than 50% | fail |
| Comparative Example 64 | pure water → comparative liquid 11 → pure water → drying | less than 50% | fail |
| Comparative Example 65 | pure water → comparative liquid 12 → pure water → drying | less than 50% | fail |

*¹ collapse suppression ratio = ( (number of cylindrical hollows not collapsed)/(total number of cylindrical hollows)) × 100 (%)

### INDUSTRIAL APPLICABILITY

The processing liquid of the present invention may be used favorably for suppressing pattern collapse of a fine metal structure, such as a semiconductor device and a micromachine (MEMS).

### DESCRIPTION OF THE SYMBOLS

101 photoresist
102 silicon oxide
103 silicon nitride
104 silicon substrate
105 circular opening
106 cylindrical hole
107 metal (titanium nitride, tungsten, hafnium oxide, tantalum or titanium)
108 cylindrical hollow of metal (titanium nitride, tungsten, hafnium oxide, tantalum or titanium)

1. A processing liquid, comprising at least one member selected from the group consisting of an ammonium halide having a fluoroalkyl group, a betaine having a fluoroalkyl group, and an amine oxide having a fluoroalkyl group.

2. The processing liquid according to claim 1, wherein a content of the at least one member is from 10 ppm to 50%.

3. The processing liquid according to claim 1, further comprising water.

4. The processing liquid according to claim 1, wherein:
the processing liquid is suitable for suppressing pattern collapse of a fine metal structure; and
a pattern of the fine metal structure comprises at least one material selected from the group consisting of titanium nitride, tungsten, hafnium oxide, tantalum and titanium.

5. A method for producing a fine metal structure, the method comprising, after wet etching or dry etching a structure, rinsing the structure with the processing liquid according to claim 1 to obtain a fine metal structure.

6. The method according to claim 5, wherein the fine metal structure comprises at least one material selected from the group consisting of titanium nitride, tungsten, hafnium oxide, tantalum and titanium.

7. The method according to claim 5, wherein the fine metal structure is a semiconductor device or a micromachine.

8. The processing liquid according to claim 1, which is suitable for suppressing pattern collapse of a fine metal structure.

9. The processing liquid according to claim 2, further comprising water.

10. The processing liquid according to claim 2, wherein:
the processing liquid is suitable for suppressing pattern collapse of a fine metal structure; and
a pattern of the fine metal structure comprises at least one material selected from the group consisting of titanium nitride, tungsten, hafnium oxide, tantalum and titanium.

11. The processing liquid according to claim 3, wherein:
the processing liquid is suitable for suppressing pattern collapse of a fine metal structure; and
a pattern of the fine metal structure comprises at least one material selected from the group consisting of titanium nitride, tungsten, hafnium oxide, tantalum and titanium.

12. A method for producing a fine metal structure, the method comprising, after wet etching or dry etching a structure, rinsing the structure with the processing liquid according to claim 2 to obtain a fine metal structure.

13. A method for producing a fine metal structure, the method comprising, after wet etching or dry etching a structure, rinsing the structure with the processing liquid according to claim 3 to obtain a fine metal structure.

14. The method according to claim 12, wherein the fine metal structure comprises at least one material selected from the group consisting of titanium nitride, tungsten, hafnium oxide, tantalum and titanium.

15. The method according to claim 13, wherein the fine metal structure comprises at least one material selected from the group consisting of titanium nitride, tungsten, hafnium oxide, tantalum and titanium.

16. The method according to claim 6, wherein the fine metal structure is a semiconductor device or a micromachine.

17. The processing liquid of claim 2, comprising the ammonium halide having a fluoroalkyl group.

18. The processing liquid of claim 2, comprising the betaine having a fluoroalkyl group.

19. The processing liquid of claim 2, comprising the amine oxide having a fluoroalkyl group.
